(19) United States
(12) Patent Application Publication
(10) Pub. No.: US 2006/0135563 A1
(43) Pub. Date: Jun. 22, 2006

(54) Method for treating HIV infection through co-administration of tipranavir and SCH-417690

(75) Inventors: Michael Friedrich Kraft, Mainz (DE); Douglas Lytle Mayers, Newtown, CT (US)

Correspondence Address: MICHAEL P. MORRIS;BOEHRINGER INGELHEIM CORPORATION, 900 RIDGEBURY ROAD, P O BOX 368, RIDGEFIELD, CT, 06877-0368, US

(73) Assignee: Boehringer Ingelheim International GmbH, Ingelheim (DE)

(21) Appl. No.: 11/280,995

(22) Filed: Nov. 17, 2005

Related U.S. Application Data
(60) Provisional application No. 60/632,561, filed on Dec. 1, 2004.

(51) Int. Cl. A61K 31/4433

(52) U.S. Cl. 514/336

(57) ABSTRACT

A method for treating HIV infection through co-administration of tipranavir and SCH-417690.

### CROSS-REFERENCE TO RELATED APPLICATIONS

Benefit of U.S. Provisional Application Ser. No. 60/632,561 filed on Dec. 1, 2004 is claimed.

### BACKGROUND OF THE INVENTION

1. Technical Field

The present invention relates to a method for treating HIV infection through co-administration of tipranavir and SCH-417690.

2. Background Information

Tipranavir (also known as PNU 140690) is a non-peptidic HIV protease inhibitor which is useful for the treatment of HIV infection. Tipranavir has the following structural formula, 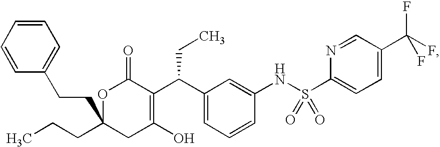
and is known by the following chemical names:
2-Pyridinesulfonamide, N-[3-[(1R)-1-[(6R)-5,6-dihydro-4-hydroxy-2-oxo-6-(2-phenylethyl)-6-propyl-2H-pyran-3-yl]propyl]phenyl]-5-(trifluoromethyl)-(Preferred CA INDEX NAME)
2-Pyridinesulfonamide, N-[3-[1-[5,6-dihydro-4-hydroxy-2-oxo-6-(2-phenylethyl)-6-propyl-2H-pyran-3-yl]propyl]phenyl]-5-(trifluoromethyl)-, [R-(R*,R*)]-(Other CA INDEX NAME)
3′-[(1R)-1-[(6R)-5,6-Dihydro-4-hydroxy-2-oxo-6-phenylethyl-6-propyl-2H-pyran-3yl]propyl]-5-(trifluoromethyl)-2-pyridinesulfonanilide (USP Dictionary of USAN and International Drug Names, 2004 Ed.).

The synthesis of tipranavir and the manner in which it may be used to treat HIV infection are described in U.S. Pat. No. 5,852,195 and published International Application WO9530670.

SCH-417690, also known as Sch-D or Schering-D, is a known per se CCR5 receptor antagonist and is useful for the treatment of HIV infection. The chemical structure of SCH-417690 is 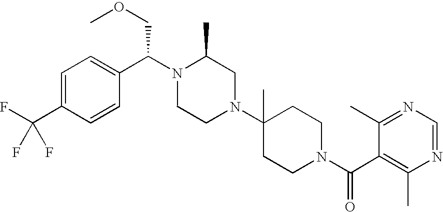
and its chemical name is 1-[(4,6-dimethyl-5-pyrimidinyl)carbonyl]-4-[4-(2-methoxy-1(R)-4-trifluoromethyl)-phenyl)ethyl-3(S)-methyl-1-piperazinyl]-4-methylpiperidine. The synthesis of SCH-417690 and the manner in which it may be used to treat HIV infection are described in and published International Application WO03084950 and published U.S. application US2004024217.

Ritonavir is an HIV protease inhibitor. Chemically it is ((2S,3S,5S)-5-(N-(N-((N-Methyl-N-((2-isopropyl-4-thiazoly)methyl)amino)carbonyl)valinyl)amino)-2-(N-((5-thiazoly)methoxycarbonyl) amino)-1,6-diphenyl-3-hydroxyhexane). It has the following structural formula.

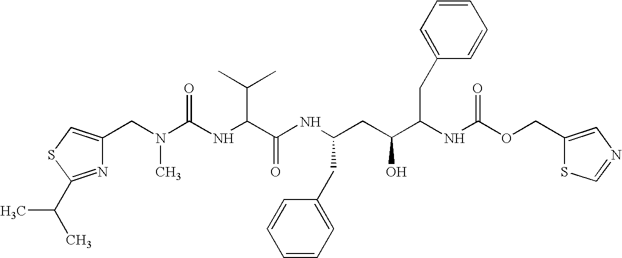

Ritonavir is currently marketed only by Abbott Laboratories, as Norvir® capsules and oral solution. The synthesis of Ritonavir is described by U.S. Pat. No. 5,541,206 and granted European Patent EP 0 674 513 B1. Ritonavir is a known inhibitor of Cytochrome P450 monooxygenase (hereinafter called “CYP”). While not approved for this purpose, ritonavir can thus be used to improve the pharmacokinetics of other drugs which are metabolized by CYP. Such use is described by U.S. Pat. No. 6,037,157 and the corresponding WO9701349. The use ritonavir for the purpose of improving the pharmacokinetics of tipranavir is described in U.S. Pat. No. 6,147,095 and the corresponding WO0025784.

### BRIEF SUMMARY OF THE INVENTION

The invention provides an improved method for the treatment of HIV infection, especially infection by HIV-1, wherein tipranavir and SCH-417690 are co-administered. The invention further comprises pharmaceutical compositions comprising both tipranavir and SCH-417690 in a single dosage form.

### DETAILED DESCRIPTION OF THE INVENTION

In accordance with the invention, a patient suffering from HIV infection, especially infection by HIV-1, is treated for such infection by means of the co-administration of tipranavir and SCH-417690, optionally in further co-administration with additional anti-viral agents.

For the purpose of carrying out the invention, tipranavir and SCH-417690 may be co-administered by way of separate dosage forms or they may optionally be combined in a single dosage form and administered simultaneously by this means.

Preferably, in accordance with the invention, tipranavir is co-administered not only with SCH-417690 but also with an inhibitor of Cytochrome P450 monooxygenase (hereinafter called “CYP”). The amount of the CYP inhibitor administered should be sufficient to inhibit the metabolism of tipranavir by CYP and thereby facilitate attainment of a therapeutically effective blood level of tipranavir. The preferred CYP inhibitor for this purpose is ritonavir, which may be employed in the manner described by U.S. Pat. No. 6,147,095 and the corresponding WO0025784.

The invention also includes pharmaceutical compositions comprising both tipranavir and SCH-417690, optionally in further combination with a CYP inhibitor, preferably ritonavir, as a single dosage form. The invention further includes is a kit of parts comprising at least two dosage forms, one comprising tipranavir and the other SCH-417690, wherein the kit optionally further includes a third dosage form comprising a CYP inhibitor, preferably ritonavir.

Those skilled in the art will know how to formulate tipranavir, SCH-417690 and CYP inhibitors, particularly ritonavir, into appropriate pharmaceutical dosage forms. Examples of the dosage forms include oral formulations, such as tablets or capsules, or parenteral formulations, such as sterile solutions.

For tipranavir, the most convenient and therefore preferable route of administration will be the oral route. Dosage forms suitable for the oral administration of tipranavir are known per se, having been described by U.S. Pat. No. 5,852,195 and published International Application WO9530670. Exemplary fill formulations for soft gelatin capsules are described by U.S. Pat. No. 6,231,887, WO9906024, WO9906043 and WO9906044.

When tipranavir is to be administered orally, an effective amount is from about 0.1 mg to 100 mg per kg of body weight per day. For adults, the preferred orally-administered dose of tipranavir is 500 mg, co-administered with 200 mg low-dose ritonavir, twice daily. Commercially available ritonavir, such as that sold by Abbott Laboratories under the brand name Norvir®, may be used.

For SCH-417690, the most convenient and therefore preferable route of administration will also be the oral route. Dosage forms suitable for the oral administration of SCH-417690 are known per se, having been described by published International Application WO0066559 and WO0066558. Clinical experience with this drug has been described at http://www.aidsmeds.com/drugs/SCH-417690.htm. In general, for the purpose of practicing the present invention, an effective orally-administered dosage of SCH-417690 would be between 5 and 15 mg QD.

The exact route of administration, dose, or frequency of administration of tipranavir (with co-administered CYP inhibitor such as ritonavir) and SCH-417690, as well as any additionally co-administered antiviral agents would be readily determined by those skilled in the art and would be dependant on the age, weight, general physical condition, or other clinical symptoms specific to the patient to be treated.

Optionally, the co-administration of tipranavir, CYP inhibitor and SCH-417690 in accordance with the invention may be accompanied by the further co-administration of additional antiviral agents. Said other antiretroviral compounds may be known antiretroviral compounds such as nucleoside reverse transcriptase inhibitors, e.g. zidovudine (3′-azido-3′-deoxythymidine, AZT), didanosine (dideoxy inosine; ddI), zalcitabine (dideoxycytidine, ddC) or lamivudine (3′-thia-2′-3′-dideoxycytidine, 3TC) and the like; non-nucleoside reverse transciptase inhibitors such as suramine, pentamidine, thymopentin, castanospermine, efavirenz, dextran (dextran sulfate), foscarnet-sodium (trisodium phosphono formate), nevirapine (11-cyclopropyl-5,11-dihydro-4-methyl-6H-dipyrido[3,-2-b: 2′,3′-e][1,4]diazepin-6-one), tacrine (tetrahydroaminoacridine) and the like; compounds of the TIBO (tetrahydro-imidazo[4,5,1-jk][1,4]-benzodiazepine-2(1H)-one and thione)-type e.g. (S)-8-chloro-4,5,6,7-tetrahydro-5- -methyl-6-(3-methyl-2-butenyl)imidazo-[4,5,1-jk][1,4]benzodiazepine-2(1H)-thione; compounds of the .alpha.-APA (.alpha.-anilino phenyl acetamide) type e.g. .alpha.-[(2-nitro-phenyl)amino]-2,6-dichlorobenzene-acetamide and the like; TAT-inhibitors, e.g. RO-5-3335 and the like; protease inhibitors e.g. indinavir, saquinovir, ABT-378 and the like; or immunomodulating agents, e.g. levamisole and the like.

1. An improved method for the treatment of HIV infection which comprises the coadminstration of tipranavir and SCH-417690.
